(19) United States
(12) Patent Application Publication
(10) Pub. No.: US 2023/0118546 A1
(43) Pub. Date: Apr. 20, 2023

(54) High-definition labeling system for medical imaging AI algorithms

(71) Applicant: PaxeraHealth Corp, Newton, MA (US)

(72) Inventors: Mohamed Shoura, Needham, MA (US); Omar Mhanna, Newton, MA (US)

(73) Assignee: PaxeraHealth Corp, Newton, MA (US)

(21) Appl. No.: 17/969,318

(22) Filed: Oct. 19, 2022

Related U.S. Application Data
(60) Provisional application No. 63/257,424, filed on Oct. 19, 2021.

(51) Int. Cl. G06V 10/778; G06T 7/00; G06V 10/764; G06V 10/94; G06V 10/776; G06V 20/70; G06V 10/82; G06T 7/10; G06V 10/26; G16H 30/40

(52) U.S. Cl. CPC G06V 10/7788; G06T 7/0012; G06V 10/764; G06V 10/945; G06V 10/776; G06V 20/70; G06V 10/82; G06T 7/10; G06V 10/26; G16H 30/40; G06V 2201/03; G06T 2207/20081; G06T 2207/20084; G06T 2207/30096; G06T 2207/30068; G06T 2207/20101

(57) ABSTRACT

An authoring tool and method by which users (e.g., diagnosticians) are enabled to design, train, and deploy custom-made AI models tailored to their needs and specific to their data. In the approach herein, and using the authoring tool, users are provided the ability to provide (feed) actual labeling to the AI during the model training process itself (i.e., prior to validation testing of the model results themselves), preferably via a master template (or “questionnaire”) that is specific to a single modality-single body part pair.

### BACKGROUND

### Technical Field

This application relates generally to information retrieval methods and systems and, in particular, to multi-layer labeling and curation of medical images that can be used for producing high performance AI imaging algorithms to deduct sophisticated radiomics.

### Background of the Related Art

Supervised learning is the machine learning task of inferring a function from labeled training data. The training data consist of a set of training examples. In supervised learning, typically each example is a pair consisting of an input object (typically a vector), and a desired output value (also called the supervisory signal). A supervised learning algorithm analyzes the training data and produces an inferred function, which can be used for mapping new examples. An optimal scenario allows for the algorithm to correctly determine the class labels for unseen instances. This requires the learning algorithm to generalize reasonably from the training data to unseen situations.

For supervised learning, the following steps are used. An initial determination is what kind of data is to be used as a training set. The training set is then gathered. In particular, a set of input objects is gathered and corresponding outputs are also gathered, either from human experts or from measurements. Then, an input feature representation of the learned function is determined. In this approach, typically the input object is transformed into a feature vector, which contains a number of features that are descriptive of the object. The structure of the learned function and corresponding learning algorithm are then determined. For example, support vector machines or decision trees may be used. The learning algorithm is then run on the gathered training set. Some supervised learning algorithms require a user to determine certain control parameters. These parameters may be adjusted by optimizing performance on a subset (called a validation set) of the training set, or via cross-validation. The accuracy of the learned function is then evaluated. After parameter adjustment and learning, the performance of the resulting function is measured on a test set that is separate from the training set.

Generalization refers to an AI model's ability to adapt properly to previously unseen data drawn from the same distribution as the original data used to create and train the model. Generalizability error (also known as out-of-sample error) is the main concern when deploying any AI system, such as a healthcare medical imaging system used by radiologists to facilitate diagnosis of mammography, x-ray chest and CT brain studies, among others. In known AI-based systems of this type, it is known to use so-called validation data sets to evaluate whether an ML model's performance is satisfactory according to some established metric; if so, the ML model is then deployed into production.

Artificial intelligence (AI)-based algorithms for medical imaging reduce medical errors and can result in large cost savings. That said, production of such algorithms typically is not secure and often requires significant time and expense, as well as coding.

### BRIEF SUMMARY

The subject matter herein provides for an authoring tool and method by which users (e.g., diagnosticians) are enabled to design, train, and deploy custom-made AI models tailored to their needs and specific to their data. In the approach herein, and using the authoring tool, users are provided the ability to provide in-depth multi-layered labeling to the AI model during the training process itself (i.e., prior to validation testing of the model results themselves), preferably via a master template (or “questionnaire”) that is specific to a single imaging machine-single body part pair. This multi-layered labeling is referred to herein as “high-definition” labeling. The imaging machine (a “modality”) and body part are selected from a predefined list, and the master template preferably has a unique identifier (or name). Typically, once the modality is selected, body parts are identified according to the selected modality. A given master template typically includes at least one question and/or one lesion template. A question typically requests non-localized information (e.g., “do you see any radiological signs of Tuberculosis”), whereas lesion templates typically seek specific localized information, e.g., that prompt the user to draw on images (e.g., an x-ray) and provide some information identifying a region-of-interest (ROI).

Information for a specified modality-body part and obtained from the authoring tool is captured as a multi-layered data set that is then selectively exposed during other imaging studies carried out by one or more users. As the one or more users perform their studies, the multi-layered data set is used to capture high definition labeling of those images by the one or more users. These interactions generate multi-layered labeled data sets. Once validated, these data sets are then used to generate or augment the training of a high performance AI imaging algorithm for detection of sophisticated radiological abnormalities or other features of interest. The AI imaging algorithm is then deployed for this purpose. Because the AI imaging algorithm is based at least in part on the multi-layered data set and the users' interactions with that data set to generate the labeling, the resulting AI algorithm is self-directed in that it leverages the users' own knowledge base and expertise. In this manner, the solution herein provides for a Do-It-Yourself (DIY) authoring platform that requires little or no coding to produce AI algorithms for all possible modalities, body parts, and anomalies. Using modeling, annotation and native data curation and auto-segmentation tools, users are empowered to securely self-develop algorithms quickly and without extensive coding.

The foregoing has outlined some of the more pertinent features of the subject matter. These features should be construed to be merely illustrative. Many other beneficial results can be attained by applying the disclosed subject matter in a different manner or by modifying the subject matter as will be described.

### BRIEF DESCRIPTION OF THE DRAWINGS

For a more complete understanding of the subject matter and the advantages thereof, reference is now made to the following descriptions taken in conjunction with the accompanying drawings, in which:

FIG. 1 is a block diagram depicting an information retrieval system in which the technique of this disclosure may be implemented;

FIG. 2 depicts the platform of this disclosure in additional detail;

FIG. 3 depicts a representative workflow according to this disclosure;

FIG. 4 depicts a set of master templates generated for a particular customer of the platform;

FIG. 5 depicts how a customer's master templates are shared across a set of branch locations or facilities associated with the customer;

FIGS. 6-7 depicts various representative information displays associated with a master template;

FIG. 8 depicts a user dialog that is exposed to a user with respect to a lesion template for a mammogram study; and

FIGS. 9-13 depict an example use case wherein a user interacts with a patient image rendered in a viewer and is prompted to enter high definition training data for use in training an AI breast detection model according to this disclosure.

### DETAILED DESCRIPTION

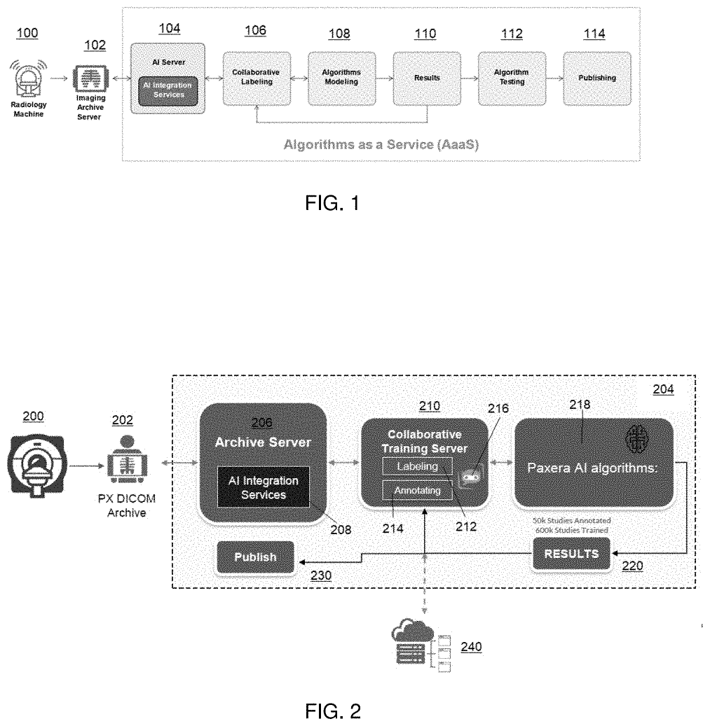

FIG. 1 depicts the basic workflow of this disclosure. In this example, a conventional radiology machine (e.g., CT, MRI, PET, X-ray, etc.) 100 provides images that are stored in an imaging archive server 102. These images are then input to a services platform 104 of this disclosure, which platform provides Algorithms-as-a-Service (AaaS) to enable platform users to collaborate for the development and use of “Do-It-Yourself” (DIY)-based AI/ML (Artificial Intelligence/Machine Learning) medical imaging algorithms. In operation, the platform 104 comprises an AI server 106 that provides AI integration services, and that enables the AaaS operations, namely, collaborative learning 108, algorithms modeling 110, the generation of results 112 (the actual models), algorithm testing 114, and publishing of the final models 116. To facilitate these operations, the platform exposes (to a set of authorized users) a set of authoring tools, and these authoring tools enable the users to collaboratively label images in a manner that provides training data for the algorithm under self-development. In this manner, and in a preferred embodiment, multiple users collaborate to pool their expertise and experience into labels that are then used to facilitate training of the model. Once the model is trained and validated, it is then published for use going forward. As will be described below, the platform provides high definition labeling technology that enables users to define multiple labeling tags, including general descriptions, image segmentation, and EMR (Electronic Medical Record) data feeds. This high definition labeling technology reduces the amount of data required for training and increases the efficiency of the created AI algorithms.

In the preferred embodiment, the platform is accessible as a service and thus multiple independent enterprises (e.g., hospitals, health care facilities, offices, labs, etc.) can utilize the authoring tools to self-develop their own medical imaging models. These models can be shared across enterprises. This deployment architecture is not intended to be limiting, as the approach herein can also be implemented in a standalone (or private) manner.

FIG. 2 depicts the technique of this disclosure in additional detail. As in FIG. 1, diagnostic image capture machines 200 capture images (e.g., PX DICOM-based images) that are then saved in an archive 202. The platform 204 of this disclosure provides imaging collaborative learning across a balanced user base dataset that helps radiologists (and other diagnosticians or interested persons) to diagnose using native AI algorithms. In particular, the platform 204 provides advanced authoring tools that enable healthcare systems, academic centers, and others to build (train) their own AI algorithms, in part using studies that they themselves label using those authoring tools. As will be described, the tools and techniques herein enable the platform, which preferably operates on a shared basis, to build anonymized dataset cohorts, and to create tagging forms (questionnaires) classified by body part to facilitate the model training, and to enable use of those forms for labeling. The platform also performs data analysis and modeling to match an appropriate model with the dataset and thus facilitates the creation and publishing of a deployed AI system. To that end, the platform 204 comprises an archive server 206 that supports AI integration services 208, and a collaborative training server 210 that facilitates the labeling 212 and annotating 214 of a model 216. One or more “stock” or native AI algorithms 218 may be provided with the platform, and these algorithms may then be customized for a particular user (or group) using the collaborative training server and based on the high definition data labeling. Once results 220 are validated, a model is then “published” or deployed into production 222.

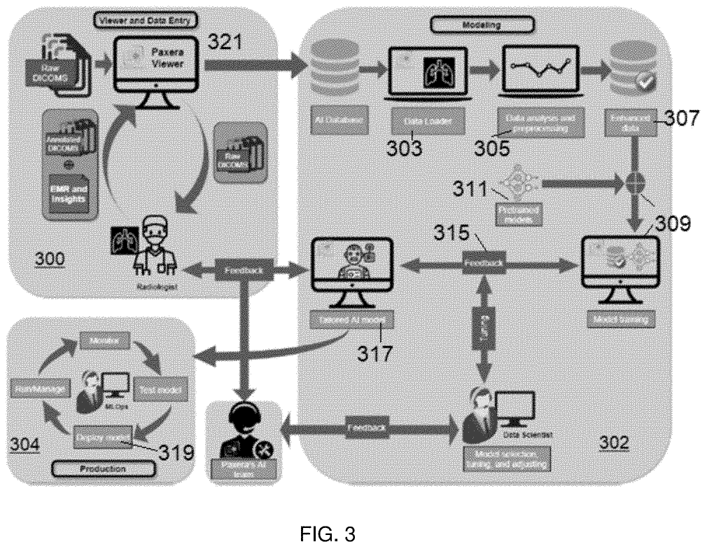

FIG. 3 provides additional details of the platform, which provides for a semi-automated ML environment to enable users the ability to design, train, and deploy their custom-made AI models tailored to their needs and specific to their data. In this representative embodiment, the system comprises three main blocks: viewer and data entry 300, modeling 302, and production 304. Typically, the user (or, more generally, the platform customer or “client”) has an on-site data scientist, an ML Ops/DevOps engineer, and one or more Radiologist (or annotated data generated by such an individual). The viewer and data entry block 300 is a module that receives raw DICOM files, and that displays them for the Radiologist. The Radiologist annotates the images using a viewer and annotation tool, preferably guided by a modality-body part template(s) as described in more detail below. The Radiologist also typically provides clinical insights. The annotated image may also be associated with relevant diagnostic information such as obtained from the patient's Electronic Medical Record (EMR) (e.g., if the patient is a smoker or has a family history of lung disease). The modeling block 302 retrieves labeled data from an AI database, e.g., using a data loader 303, and performs preliminary analysis and pre-processing 305 (e.g., data cleaning or normalization) to enhance quality of the dataset 307. Modeling 309 is then performed on the enhanced dataset, e.g., using one or more pretrained models 311. Fine tuning 313 of the model on the enhanced data is performed, e.g., by adjusting hyperparameters of the model. The process of training and evaluating the model based on feedback 315 is repeated until one or more set benchmarks are achieved. In the production block 304, the tailored AI model 317 is evaluated and deployed 319. The process of managing and monitoring the modules continues until a next version is ready for deployment.

To facilitate the machine learning, the platform provides an authoring tool 321 in the form of a web-based editor from which one or more master templates are built. As noted above, and according to this disclosure, users are provided the ability to provide (feed) actual labeling to the AI during the model training process itself (i.e., prior to validation testing of the model results themselves), preferably via a master template (or “questionnaire”) that is specific to a single modality-single body part pair. To generate a master template, the modality and body part are selected from a predefined list, and the master template is provided a unique identifier (or name). Typically, once the modality is selected, body parts are identified according to the selected modality. A given master template typically includes at least one question and/or one lesion template. A question typically requests non-localized information, whereas lesion templates typically seek specific localized information, e.g., that prompt the user to draw on images (e.g., an x-ray) and provide some information identifying a region-of-interest (ROI).

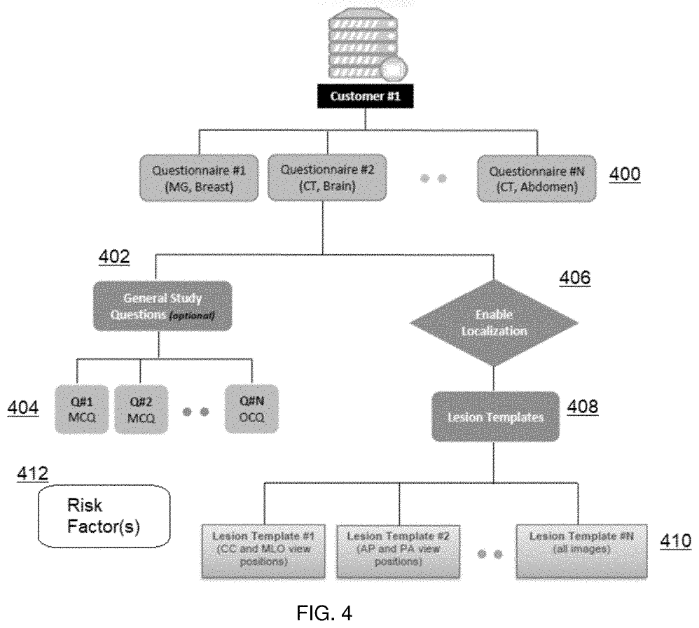

As depicted in FIG. 4, preferably master templates 400 are saved per service provider (platform) customer 402. A particular master template, e.g., for the {CT, Brain} modality/body part pairing may comprise multi-layer criteria such as General Study Questions 402 and, if Localization is enabled at 406, a set of one or more Lesion Templates 408. Typically, the notion of Localization refers to the identification of one or more particular regions of interest (ROI) in a rendered image of a study. General Study Questions thus seek non-Localized information, and there may be one or more such questions 404. Lesion Templates 410 typically vary by distinct view positions, e.g., CC and MLO view positions for Lesion Template #1, AP and PA view positions for Lesion Template #2, and so forth. A template may also include one or more Risk Factors 412, e.g., information derived from a patient EMR.

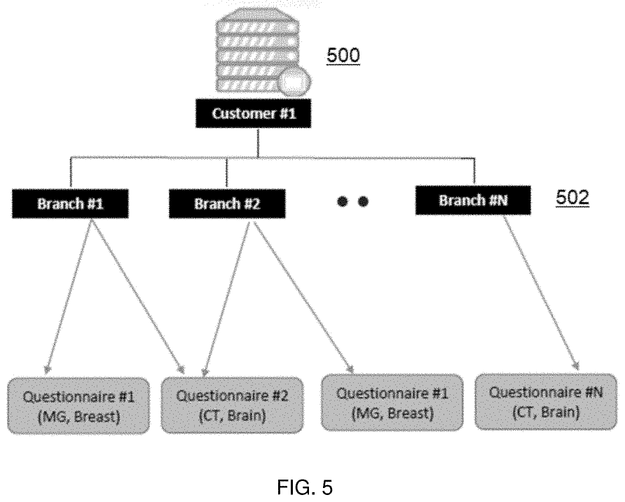

As depicted in FIG. 5, assume that a service provider 500 has a number of branches, e.g., a set of hospitals in a hospital system. Each branch 502 of the customer can share the same master template, and preferably all master templates are saved (e.g., in a JSON file, per HTTPS-based request and response semantics) in a customer-specific platform directory. As also depicted in FIG. 5, each branch can choose among many questionnaires that have been created for the customer. Preferably, the platform creates a new JSON file in each branch directory and that contains an identifier of the linked questionnaire(s). Preferably, the branch JSON file is updated when a particular questionnaire is deleted for the customer. Once a user opens a viewer for any study, and based on customer, branch, modality and body part, then an available and responsive questionnaire is returned as a JSON object.

According to this disclosure, the platform exposes a web-based Questionnaire to enable a user (or user group) to custom build questions that pertain to diagnostic criteria of a particular clinical feature of interest, typically an abnormality such as a mass, a lesion, a cyst, or the like. A clinical feature of interest is sometimes referred to herein as a region of interest (ROI). A Questionnaire is sometimes referred to herein as a template. A template exposes a set of information fields that define multi-layered criteria for diagnosing the clinical feature of interest. Typically, the information fields solicit one or more of the following “layers” of information with respect to an image, namely, an answer to a general (i.e., non-localized) question about the clinical feature of interest, a prompt for the user to draw localized information on the image and to enter accompanying descriptive information identifying the ROI, and information about a patient risk factor. Some information, such as the patient risk factor data, may be obtained directly or programmatically from other data sources (e.g., EMRs). During provisioning, a template is configured by specifying the information fields and the data to be captured by those fields. As will be described, the configured template for the clinical feature of interest is sometimes referred herein as a multi-layered data set. The system then selectively exposes the configured template as other users evaluate imaging studies for the clinical feature of interest. As those other users interact with the configured template and, in particular, as they review the radiographic findings and examine morphological features, the users are prompted by the configured template to enter information specific to what they are viewing. As a result, and for each such interaction, a multi-layered labeled data set for the clinical feature of interest is generated. The system then collects these multi-layered labeled data sets and uses them to train an AI algorithm. Once trained, that AI algorithm is then used to classify new images for the feature of interest.

In one embodiment, the Questionnaire is defined by an administrator and then used by a set of clinicians (the users). A service provider may publish (make available) a predefined or configured set of templates from a template repository. A particular entity (e.g., a hospital) or entity location (a regional branch of a hospital, a working group, or the like) may define its own Questionnaire; thus, the nature of the particular ML model that is generated from the data captured from a particular Questionnaire may be global or location specific, entity-specific, department-specific, and the like. As noted above, the information solicited by the template enables particular radiographic findings and morphological features to be examined and annotated to facilitate the high definition training for a self-generated AI model. The Questionnaire thus enables users to define criteria that are useful to feed clinical input to an AI model.

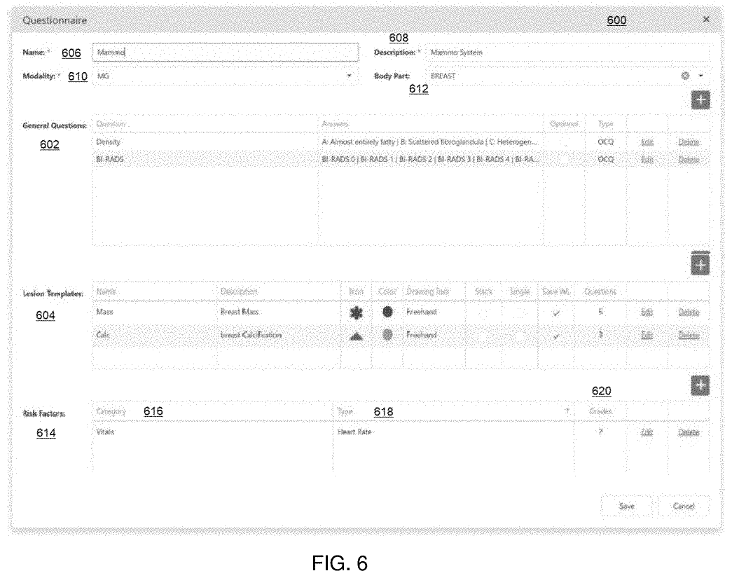

FIG. 6 depicts a representative forms-based user interface to define the Questionnaire for a specific modality and specific body part for particular disease of interest. The interface typically comprises a set of one or more configuration pages. Preferably, each Questionnaire 600 has a set of General Questions 602 and Lesion Templates 604 used to obtain information that will be later used to build an AI dataset on the fly. As shown, the Questionnaire 600 includes a set of attribute fields, e.g., Name 606, Description 608, Modality 610, Body Part 612, the list of General Questions 602, the list of Lesion Templates 604, and possibly others, such an optional list of Image Grouping criteria (not shown). As also depicted in FIG. 6, the interface typically also includes a Risk Factors section 614 that can be used to define and obtain additional information, typically based on a patient's particular background or clinical history. To that end, Risk Factors section 614 has several attributes such as Category 616 and Type 618, and Risk Factor Severity Grades may be defined using fill-in fields in a Severity Grade table 620.

Generalizing, the configuration pages that comprise the Questionnaire comprise a substrate for mapping out the work of image classification, vision segmentation and labeling for a particular ML algorithm of interest. Using the custom fields, appropriate selections are entered to define the diagnostic criteria for the clinical feature of interest and that is being defined by the particular Questionnaire. The nature and type of information that are defined/selected by the user will depend on the clinical feature. In an example, assume that the feature of interest is breast cancer. In this example, the user may enter “Mammography for breast cancer” in the Name field and enter an appropriate description of the algorithm in the Description field 608. Typically, the Name field is unique. The modality and body part are selected from predefined dropdown lists. Modality typically refers to a type of imaging machine, e.g., CT, MRI, US (ultrasound), MG (mammogram), etc.). Conveniently, multiple modality data sets can be assigned to the algorithm. Thus, for example, here the user may select both mammogram (MG) and ultrasound studies from the dropdown list for Modality 610. The user enters “breast” in the Body Part field for this example of course. The Questionnaire 600 includes at least one question (configured in General Question field 602) or one Lesion Template (configured in Lesion Template field 604), and typically there are multiple questions and multiple lesion templates. The inclusion of a Lesion Template enables the user to draw on images and mark regions of interest (ROI). General Questions typically involve the user just answering a question without drawing (i.e., no localization on a particular image). Typically, each Questionnaire exposes several possible combinations of prompting: an optional list of General Questions (e.g., Do you see any radiological signs of Tuberculosis?) and an optional list of Lesion Templates that should be drawn on a specific image in a study (e.g., draw lesion with specific attributes on a given view position of a chest x-ray image). Preferably, the system configurator does not allow the administrator or other user to provision multiple questionnaires for the same modality and same body part. In other words, a Questionnaire for a modality-body part pair is unique. Preferably, entries in the Modality and Body Part fields are mandatory. Once the Modality is selected from the dropdown, the Body Parts are filled according to the selected Modality. As noted above, a user can optionally add one or more Image Grouping Criteria (e.g., attribute name and supported values) from several options, e.g., image view position, image laterality, image type (e.g., the user can select an attribute name=image laterality with L and R supported values). Preferably, the user is prevented from adding grouping criteria with the same attribute. More generally, the user (or at least an authorized user, such as an administrator) has the ability to add, edit or delete a Questionnaire. Preferably, the system confirms the user's intention (e.g., via a prompt) when he or she is attempting to delete a template that is currently linked with branches to one or more other templates.

Referring back to the General Questions field, preferably each question has a specific type, such as MCQ (multiple choice), OCQ (one choice), Polar (yes/no) and Fill In (answer is free text). Preferably, a question has the following attributes: Question Text, Question Type (MCQ,OCQ, etc.), and whether the question or Mandatory or Optional (default is Mandatory).

Question details typically are defined according to the Question Type. For MCQ and OCQ questions, each question must have one more possible answers, and preferably the system affords the user the ability to delete or edit a specific answer. For Polar and Fill-In questions, no specific details are required.

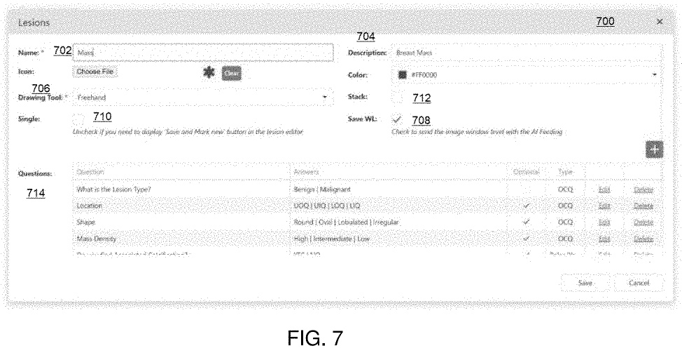

Selection of an entry in the Lesion Templates page navigates the user to a linked Lesions page 700, such as depicted in FIG. 7. Generalizing, a lesion template defines a way to specify a region of interest on a medical image by drawing or annotation. To this end, this linked page includes a set of attributes for each Lesion Template, namely Name 702, Description 704, a field 706 to designate a Drawing Tool type (e.g., polyline, ellipse or rectangle), a checkbox 708 to enable storing of the image (WL for this lesion), a Single 710 or Stack 712 checkbox to enable adding more than one lesion on the same image, and a set of Questions 714 conforming to the various types (MCQ, OCQ, Polar, Fill-In). In this example, the Questions include Lesion Type (mandatory), and the following optional Questions: Location, Shape, Mass Density, the existence of any associated Calcification, and so forth. Preferably, the lesion Name is unique. The user can add multiple Lesion Templates, e.g., one template for a Mass lesion, and another template for Calcification Lesion, and so forth. If a Questionnaire has at least one mark Lesion Template, localization is enabled. Users can optionally add one or more image matching Criteria (attribute name and values) from the following: image view position, image laterality, and image type. Each imaging Criteria has predefined values filled based on the modality of the parent template, e.g., CC, MLO . . . for the Mammogram (MG) questionnaire, AP, PA . . . for DX questionnaire, and so forth. If no imaging criteria are defined for the lesion, the user can mark the lesion with template on any image in the study.

Lesion Templates enables classes of anomalies (mass, cyst, calcification, architectural distortion, etc.) to be segmented on the images displayed to the user. Each type of anomaly typically has an associated type for the segmenting tool, and typically there are multiple segmenting tools associated with a template. Using the Lesion Template, and as described above, the designer can add questions about the morphology of each lesion (e.g., lesion type, location, shape, density, associated calcification, etc.).

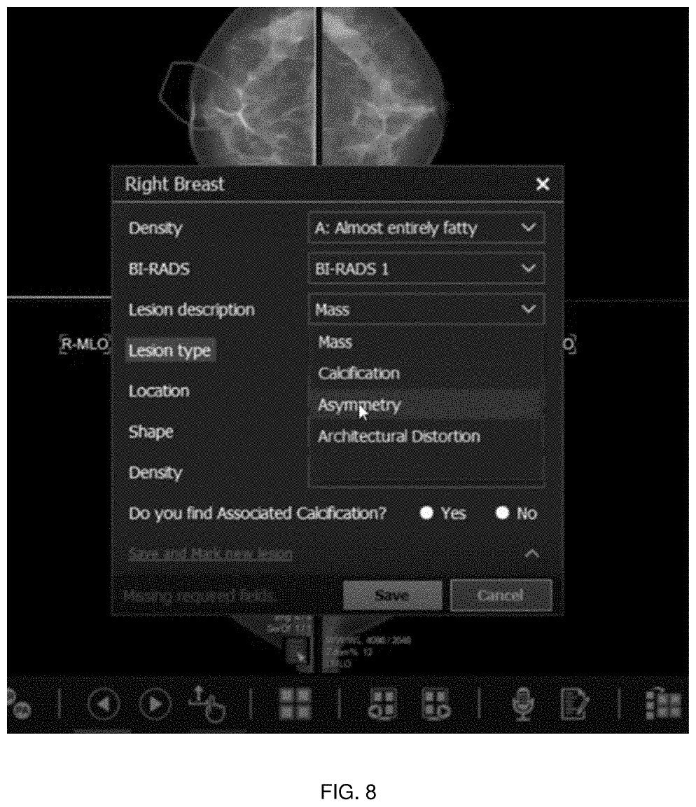

FIG. 8 depicts a representative window that is then exposed to the user to collect this information during a user's particular interaction, in this case a review of a mammogram study. As noted above, the collected information comprises a multi-layer labeled data set derived from the particular image that is being viewed in the study. In this example, the user viewing the study image has identified the breast lesion has Density “Almost entirely fatty,” identified the Lesion description as a “Mass,” and is in the process of providing additional labeling about the particular clinical feature of interest in this particular study. If the Risk Factors portion of the template is also configured, the designer can add further information of this type to further enhance the model's results. As noted above, typically Risk Factor data is extracted from individual patient EMRs. The information may be entered directly, or programmatically. Typically, risk factor data includes patient age, history, medications, laboratory results data, race, and the like. To facilitate access to such data, the system preferably integrates with one or more EMR or other similar systems.

As noted above, the platform of this disclosure enables ML algorithms to be developed in a DIY manner. A representative field of use is for radiology, although this is not a limitation. The platform enables researchers, clinicians and data scientists to self-develop machine learning algorithms that require zero coding and that are clinically-validated. From a high level configuration page, the platform enables the designer to select (e.g., from a dropdown) a type of ML model (e.g., segmentation, classification, or the like), and to enable the model for use in the imaging system. Selection of “segmentation” type enables the user to segment lesions, and selection of the “classification” type enables classification of the image. From this configuration page, the designer can identify a user or set of users to participate in the model training, i.e., to work collaboratively, in the training by interactions with the templates. In addition, and to facilitate training image models, the platform preferably is linked with available data sources in a facility (or across facilities) so that facilities can access and use their existing data sets (e.g., images) as well as incoming or other data sets available to them.

In the example referenced above, a questionnaire is developed for an algorithm that is developed to study breast malignancy on conventional diagnostic mammograms. For example, the model is a Mask-RCNN model, which provides pixel-based segmentation of lesions in the mammogram, and in this example the model is trained on six (6) different classes: Benign Architectural Distortion, Benign Calcification, Benign Masses, Malignant Architectural Distortion, Malignant Calcification, and Malignant Masses. By way of background, CNN refers to a Convolutional Neural Network, which is an artificial neural network architecture that consists of three main layers, a convolutional layer, a pooling layer, and one or more fully connected layers. The convolutional layer abstracts an image input as a feature map via the use of filters. The pooling layer down-samples feature maps by summarizing the presence of features therein. The fully connected layers connect every neuron in one layer to every neuron in another layer. R-CNN refers to a family of CNN-based machine learning models for computer vision and specifically object detection. Given an input image, R-CNN begins by applying a mechanism called selective search to extract regions of interest (ROI), where each ROI is a rectangle that may represent the boundary of an object in image. Each ROI is fed through a neural network to produce output features. For each ROI's output features, a collection of support-vector machine classifiers is used to determine what type of object (if any) is contained within the ROI. While the original R-CNN independently computed the neural network features on each of the regions of interest, Fast R-CNN runs the neural network once on the whole image. Further, previous versions of R-CNN focused on object detection, Mask R-CNN adds the capability for instance segmentation. In this example, the high definition training data collected by the user interaction(s) with the system are utilized for training the Mask-RCNN model.

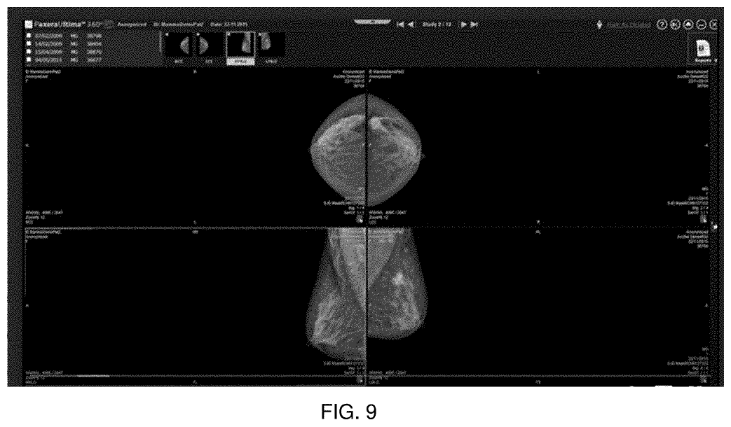

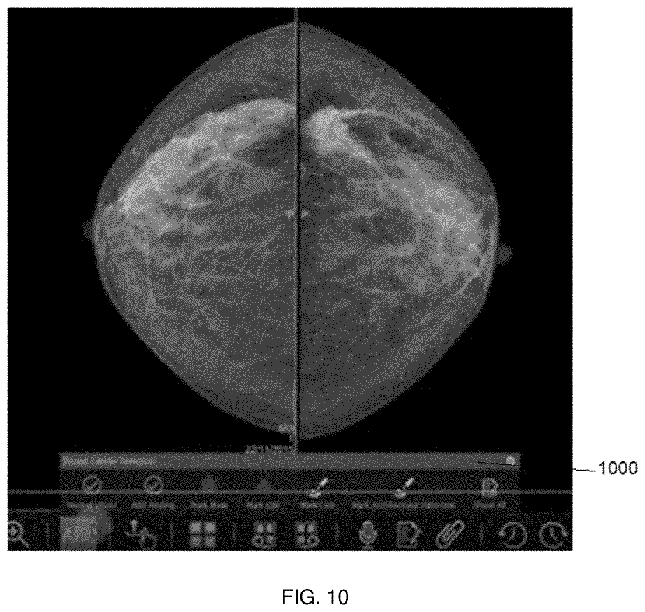

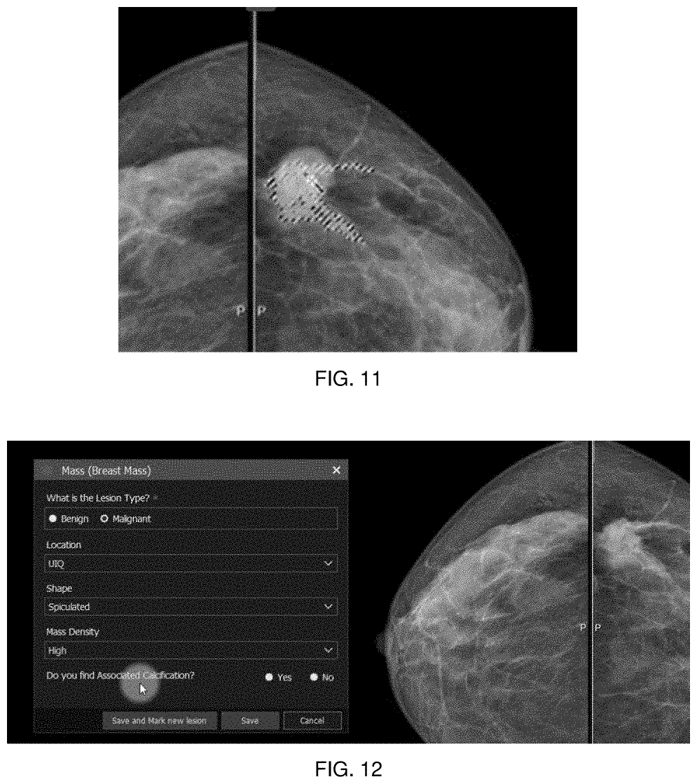

Assume now that the user (e.g. a Radiologist) has selected a study for analysis. Image rendering systems may be used for this purpose. In a preferred embodiment, the platform technologies and tools of this disclosure are integrated with the existing image rendering system, e.g., and exposed as an option for selection. To provide a concrete example, and with reference to FIG. 9, assume now that the user has begun to review left and right breast images, such as depicted in the image rendering system. The user then notices a mass in the upper inner quadrant of the patient's left breast. By selecting a control (a button, an icon, a dropdown entry, etc.) for the subject tool, a segmentation toolbar 1000 is then rendered as depicted in FIG. 10. The segmentation tool 1000 is then used to begin segmenting the mass. In this example, the user selects a Mark Mass tab in the toolbar 1000 enable the segmenting tool for this class of morphology. As the user lays down a seed point into the tissue of the mass, the entire region of the mass is automatically highlighted as depicted in FIG. 11. This results in an area of segmentation. Once segmentation has been completed, the user is then prompted to enter the high definition training data that has been configured for this type of lesion in the Questionnaire and its associated Lesion Template(s) as described above. FIG. 12 depicts this interaction for a breast mass that has a high potential for malignancy. In this example, the template exposes several defining criteria, namely, lesion type (benign or malignant), location, shape, density and existence of associated calcification. The user fills in the requested information from the dropdown options that have been configured. The criteria as specified by the user is shown in the fill-in fields, and this process generates the multi-layered labeled data set, as previously described. The clinical information and associated labeling input by the user in this manner is then fed to the breast detection model (e.g., the Mask-RCNN model) to facilitate training of that model. In a similar manner, and as noted, other users view images and provide similar inputs. In this manner, the AI model learns to detect lesions addition based on the radiometric features and the user-input high definition training data.

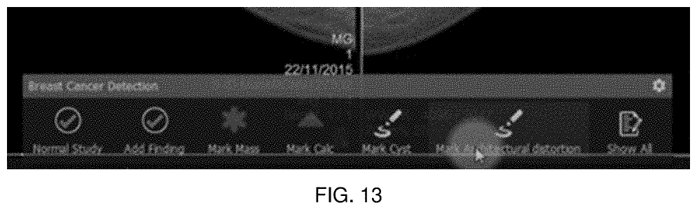

Continuing with the breast cancer detection model as the example, breast malignancy can manifest in the form of a mass, a cluster of micro-calcifications, and/or architectural distortion. Thus, in this example, the segmentation toolbar also exposes other tabs (panels) for enabling each of these anomalies to be selected as appropriate. These toolbar options are shown in FIG. 13.

As noted above, information obtained by the questionnaire may be augmented with additional relevant Risk Factor data, such as laboratory results, data from the patient's health records, and the like.

A particular template may identify multiple features of interest for labeling. Thus, for example, a first level feature of interest may be a tumor that has various associated characteristics, such as the tumor's contour. In this example, the template may also expose an additional set of questions or annotation/drawing options with respect to these additional characteristics. These template elements constitute a second level of labeling.

The techniques of this disclosure have many advantages. Self-authoring of AI-based medical imaging AI algorithms as described herein reduces patient privacy risk, reduces time and cost, and reduces or obviates extensive software coding. Imaging systems that incorporate the described technologies are more robust and efficient, as they enable automated detection of sophisticated radiomics. The techniques herein provide for improvements to such imaging technologies.

### Enabling Technologies

Typically, the computing platform (FIG. 2, 240) is managed and operated “as-a-service” by a service provider entity. In one embodiment, the platform is accessible over the publicly-routed Internet at a particular domain, or sub-domain. The platform is a securely-connected infrastructure (typically via SSL/TLS connections), and that infrastructure includes data encrypted at rest, e.g., in an encrypted database, and in transit. The computing platform typically comprises a set of applications implemented as network-accessible services. One or more applications (services) may be combined with one another. An application (service) may be implemented using a set of computing resources that are co-located or themselves distributed. Typically, an application is implemented using one or more computing systems. The computing platform (or portions thereof) may be implemented in a dedicated environment, in an on-premises manner, as a cloud-based architecture, or some hybrid.

The system (FIG. 2, 240) may be implemented on-premises (e.g., in an enterprise network), in a cloud computing environment, or in a hybrid infrastructure. An individual end user typically accesses the system using a user application executing on a computing device (e.g., mobile phone, tablet, laptop or desktop computer, Internet-connected appliance, etc.). In a typical use case, a user application is a mobile application (app) that a user obtains from a publicly-available source, such as a mobile application storefront. The platform may be managed and operated by a service provider. Although typically the platform is network-accessible, e.g., via the publicly-routed Internet, the computing system may be implemented in a standalone or on-premises manner. In addition, one or more of the identified components may interoperate with some other enterprise computing system or application.

As described above, the platform supports a machine learning system. The nature and type of Machine Learning (ML) algorithms that are used to process the query may vary. As is known, ML algorithms iteratively learn from the data, thus allowing the system to find hidden insights without being explicitly programmed where to look. ML tasks are typically classified into various categories depending on the nature of the learning signal or feedback available to a learning system, namely supervised learning, unsupervised learning, and reinforcement learning. In supervised learning, the algorithm trains on labeled historic data and learns general rules that map input to output/target. The discovery of relationships between the input variables and the label/target variable in supervised learning is done with a training set, and the system learns from the training data. In this approach, a test set is used to evaluate whether the discovered relationships hold and the strength and utility of the predictive relationship is assessed by feeding the model with the input variables of the test data and comparing the label predicted by the model with the actual label of the data. The most widely used supervised learning algorithms are Support Vector Machines, linear regression, logistic regression, naive Bayes, and neural networks. As will be described, the techniques herein preferably leverage a network of neural networks. Formally, a NN is a function g: X→Y, where X is an input space, and Y is an output space representing a categorical set in a classification setting (or a real number in a regression setting). For a sample x that is an element of X, g(x)=f_{L}(f_{L-1}( . . . ((f₁/(x)))). Each fᵢ represents a layer, and f_{L} is the last output layer. The last output layer creates a mapping from a hidden space to the output space (class labels) through a softmax function that outputs a vector of real numbers in the range [0, 1] that add up to 1. The output of the softmax function is a probability distribution of input x over C different possible output classes.

Thus, for example, in one embodiment, and without limitation, a neural network such as described is used to extract features from an utterance, with those extracted features then being used to train a Support Vector Machine (SVM).

One or more functions of the computing platform of this disclosure may be implemented in a cloud-based architecture (FIG. 2, 240). As is well-known, cloud computing is a model of service delivery for enabling on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. Available services models that may be leveraged in whole or in part include: Software as a Service (SaaS) (the provider's applications running on cloud infrastructure); Platform as a service (PaaS) (the customer deploys applications that may be created using provider tools onto the cloud infrastructure); Infrastructure as a Service (IaaS) (customer provisions its own processing, storage, networks and other computing resources and can deploy and run operating systems and applications).

The platform may comprise co-located hardware and software resources, or resources that are physically, logically, virtually and/or geographically distinct. Communication networks used to communicate to and from the platform services may be packet-based, non-packet based, and secure or non-secure, or some combination thereof.

More generally, the techniques described herein are provided using a set of one or more computing-related entities (systems, machines, processes, programs, libraries, functions, or the like) that together facilitate or provide the described functionality described above. In a typical implementation, a representative machine on which the software executes comprises commodity hardware, an operating system, an application runtime environment, and a set of applications or processes and associated data, that provide the functionality of a given system or subsystem. As described, the functionality may be implemented in a standalone machine, or across a distributed set of machines.

Other enabling technologies for the machine learning algorithms include, without limitation, vector autoregressive modeling (e.g., Autoregressive Integrated Moving Average (ARIMA)), state space modeling (e.g., using a Kalman filter), a Hidden Markov Model (HMM), recurrent neural network (RNN) modeling, RNN with long short-term memory (LSTM), Random Forests, Generalized Linear Models, Extreme Gradient Boosting, Extreme Random Trees, and others. By applying these modeling techniques, new types of features are extracted, e.g., as follows: model parameters (e.g. coefficients for dynamics, noise variance, etc.), latent states, and predicted values for a next couple of observation periods.

Typically, but without limitation, a client device is a mobile device, such as a smartphone, tablet, or wearable computing device, laptop or desktop. A typical mobile device comprises a CPU (central processing unit), computer memory, such as RAM, and a drive. The device software includes an operating system (e.g., Google® Android™, or the like), and generic support applications and utilities. The device may also include a graphics processing unit (GPU). The mobile device also includes a touch-sensing device or interface configured to receive input from a user's touch and to send this information to processor. The touch-sensing device typically is a touch screen. The mobile device comprises suitable programming to facilitate gesture-based control, in a manner that is known in the art.

Generalizing, the mobile device is any wireless client device, e.g., a cellphone, pager, a personal digital assistant (PDA, e.g., with GPRS NIC), a mobile computer with a smartphone client, or the like. Other mobile devices in which the technique may be practiced include any access protocol-enabled device (e.g., an Android™-based device, or the like) that is capable of sending and receiving data in a wireless manner using a wireless protocol. Typical wireless protocols are: WiFi, GSM/GPRS, CDMA or WiMax. These protocols implement the ISO/OSI Physical and Data Link layers (Layers 1 & 2) upon which a traditional networking stack is built, complete with IP, TCP, SSL/TLS and HTTP.

Each above-described process preferably is implemented in computer software as a set of program instructions executable in one or more processors, as a special-purpose machine.

While the above describes a particular order of operations performed by certain embodiments, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the disclosed subject matter has been described in the context of a method or process, the subject matter also relates to apparatus for performing the operations herein. This apparatus may be a particular machine that is specially constructed for the required purposes, or it may comprise a computer otherwise selectively activated or reconfigured by a computer program stored in the computer. Such a computer program may be stored in a computer readable storage medium, such as, but is not limited to, any type of disk including an optical disk, a CD-ROM, and a magnetic-optical disk, a read-only memory (ROM), a random access memory (RAM), a magnetic or optical card, or any type of media suitable for storing electronic instructions, and each coupled to a computer system bus.

A given implementation of the computing platform is software that executes on a hardware platform running an operating system such as Linux. A machine implementing the techniques herein comprises a hardware processor, and non-transitory computer memory holding computer program instructions that are executed by the processor to perform the above-described methods.

The functionality may be implemented with other application layer protocols besides HTTP/HTTPS, or any other protocol having similar operating characteristics.

There is no limitation on the type of computing entity that may implement the client-side or server-side of the connection. Any computing entity (system, machine, device, program, process, utility, or the like) may act as the client or the server.

While given components of the system have been described separately, one of ordinary skill will appreciate that some of the functions may be combined or shared in given instructions, program sequences, code portions, and the like. Any application or functionality described herein may be implemented as native code, by providing hooks into another application, by facilitating use of the mechanism as a plug-in, by linking to the mechanism, and the like.

The platform functionality (FIG. 2, 240) may be co-located or various parts/components may be separately and run as distinct functions, perhaps in one or more locations (over a distributed network).

Each above-described process preferably is implemented in computer software as a set of program instructions executable in one or more processors, as a special-purpose machine.

As previously noted, the techniques herein generally provide for the above-described improvements to a technology or technical field (e.g., medical imaging systems), as well as the specific technological improvements to various fields, all as described above.

Preferably, the authoring tool is implemented as a web-based editor tool, namely, software executing on a hardware processor.

Of course, the breast cancer detection model described above is not intended to be limiting, as the basic approach herein can be used for many other types of diseases of interest and their associated modalities. These include, without limitation, common thorax disease (DX|CR modality), intracranial hemorrhage (CT modality), acute liver failure (ALF) (CT modality), thyroid nodules (US modality), Liver tumors (MR modality), bone age assessment (DX|CR modality), liver cancer (CT modality), hand tumor assessment (CR|DX modality), focal splenic lesions (CT), Reed Sternberg Cell (OT modality), Ductal Carcinoma in situ (SM|OT modality), CT lung nodules (CT), focal sclerotic lesions (MR), cell type classification (OT|SM), and others. The AI models for these conditions of course will vary.

What is claimed is as follows:

1. A method for medical imaging, comprising:
providing a template uniquely associated with a modality-body part pair, the template exposing a set of information fields that define multi-layered criteria for diagnosing a feature of interest, the information fields soliciting at least two of: an answer to a question, definition of a region of interest on a medical image by drawing or annotation, and identification of a risk factor;
receiving information in the set of information fields, thereby configuring the template with respect to the feature of interest, the information comprising a multi-layered data set;
for each of one or more users:
as a patient medical image is rendered in a viewer, and in response to identification of the feature of interest, retrieving the configured template;
based on the configured template, receiving data entered by the user; and
in response, generating a multi-layered labeled data set for the feature of interest;
using the multi-layered labeled data sets derived from the one or more users to train a machine learning model; and
using the machine learning model to automatically classify the feature of interest for one or more additional medical images.

2. The method as described in claim 1 wherein the one or more users comprise a set of users associated with one of: a given facility, and two or more facilities.

3. The method as described in claim 1 wherein providing the template includes rendering a configuration page that exposes the set of information fields.

4. The method as described in claim 3 wherein the configuration page exposes one or more lesion templates, wherein a lesion template defines the region of interest on the medical image.

5. The method as described in claim 1 wherein the information configuring the template includes one of: an answers to the question, data defining a lesion template, and data defining the one or more risk factors.

6. The method as described in claim 1 wherein the machine learning model provides one of: classification, and segmentation.

7. The method as described in claim 1 wherein the machine learning model is a convolutional neural network (CNN).

8. The method as described in claim 1 wherein receiving data in the configured tool includes activating a segmenting tool for a particular class of pathology as represented by the feature of interest.

9. The method as described in claim 1 wherein the feature of interest is one of: a mass, a calcification and an architectural distortion.

10. The method as described in claim 1 wherein the data received in the configured template includes one of: location, shape, density, and an indication of associated calcification.

11. The method as described in claim 1 wherein the clinical feature of interest is breast cancer and the machine learning model is a Mask-RCNN model.

12. The method as described in claim 1 further including validating the machine learning model.

13. A software-as-a-service (SaaS) platform, comprising:
a set of hardware processors;
computer memory holding computer program code executed by the one or more hardware processors to train and use a machine learning algorithm for use in medical imaging, the computer program code comprising program code configured to:
provide a template uniquely associated with a modality-body part pair, the template exposing a set of information fields that define multi-layered criteria for diagnosing a feature of interest, the information fields soliciting at least two of: an answer to a question, definition of a region of interest on a medical image by drawing or annotation, and identification of a risk factor;
receive information in the set of information fields, thereby configuring the template with respect to the feature of interest, the information comprising a multi-layered data set;
for each of one or more users:
as a patient medical image is rendered in a viewer, and in response to identification of the feature of interest, retrieve the configured template;
based on the configured template, receive data entered by the user; and
in response, generate a multi-layered labeled data set for the feature of interest;
use the multi-layered labeled data sets derived from the one or more users to train a machine learning model; and
use the machine learning model to automatically classify the feature of interest for one or more additional images.

14. The SaaS platform as described in claim 13 wherein the one or more users comprise a set of users associated with one or more enterprises or facilities.

15. A computer program product in a non-transitory computer-readable medium, the computer program product comprising computer program code executable by a hardware processor to train and use a machine learning model for use in medical imaging, the computer program code configured to:
provide a template uniquely associated with a modality-body part pair, the template exposing a set of information fields that define multi-layered criteria for diagnosing a feature of interest, the information fields soliciting at least two of: an answer to a question, definition of a region of interest on a medical image by drawing or annotation, and identification of a risk factor;
receive information in the set of information fields, thereby configuring the template with respect to the feature of interest, the information comprising a multi-layered data set;
for each of one or more users:
as a patient medical image is rendered in a viewer, and in response to identification of a clinical feature of interest, retrieve the configured template;
based on the configured template, receive data entered by the user; and
in response, generate a multi-layered labeled data set for the feature of interest;
use the multi-layered labeled data sets derived from the one or more users to train a machine learning model; and
use the machine learning model to automatically classify the feature of interest for one or more additional images.
